(19) United States
(12) Patent Application Publication
(10) Pub. No.: US 2022/0098549 A1
(43) Pub. Date: Mar. 31, 2022

(54) METHOD FOR CHARACTERISING A TISSUE-ENGINEERED CONSTRUCT

(71) Applicant: 1LAB SA, Manno (CH)

(72) Inventors: Enrico PERFLER, Manno (CH); Andrea BIFFI, Manno (CH); Stefano FARINA, Manno (CH)

(21) Appl. No.: 17/429,295

(22) PCT Filed: Feb. 14, 2020
(86) PCT No.: PCT/IB2020/051266

§ 371 Date: Aug. 6, 2021

(30) Foreign Application Priority Data
Feb. 14, 2019 (IT) 102019000002193

(51) Int. Cl. C12N 5/071; G01N 33/50

(52) U.S. Cl. CPC C12N 5/069; G01N 33/5085; G01N 21/6428; C12N 2533/30; C12N 2533/50; G01N 33/5064

(57) ABSTRACT

A method for characterising a tissue-engineered construct and the tissue-engineered construct are described. The characterisation method allows verification of viability, morphology, functionality and/or distribution of cells comprised in the tissue-engineered construct. The tissue-engineered construct has a scaffold with a lumen and at least one portion of the lumen lined with at least one functional and preferably continuous cell layer, that can be used for in vitro testing of medicinal products for human or animal use. A method for the in vitro testing of medicinal products for human or animal use performed with the tissue-engineered construct is also described.

The present invention regards a method for characterising a tissue-engineered construct, comprising a scaffold having at least one portion of the lumen lined with at least one functional and preferably continuous cell layer, that can be used for the in vitro testing of medicinal products for human or animal use. In particular, said characterisation method allows to verify the viability, the morphology, the functionality and/or the distribution of the cells comprised in the tissue-engineered construct. Furthermore, the present invention regards the tissue-engineered construct characterised by means of said method and a method for the in vitro testing of medicinal products for human or animal use by means of said tissue-engineered construct.

It is known that the development of a medicinal product entails a long process. In the context of the present invention, the expression medicinal product is used to indicate any product for medical use both on humans and animals, such as a drug or a medical device or a combination thereof. Basically, in the medicinal product development process, prior to using the product in humans or in animals it is necessary to determine the type of effects on the tissue/s with which it comes into contact so as to evaluate the biological safety, the efficiency of the medicinal product and to predict potential problems relating to the use thereof.

Today, the evaluation of the biological safety and of the efficiency of a medicinal product is based on in vitro, ex vivo and in vivo tests on animal models that have significant differences with respect to the final conditions of use, in particular in humans. Such differences are observable even further when evaluating medicinal products for human or animal use that provide for the use thereof in the cardiovascular and peripheral vascular region, such as, for example, heart valves, stents, grafts, catheters, bandages, nets or filters. As a matter of fact, the evaluation of the interaction of medicinal products for human or animal use with the vascular tissues and with the blood so as to be able to establish the biological safety and efficiency thereof is crucial and the models currently in use reveal major limits and drawbacks both anatomical/structural and regarding the blood composition.

In addition, in vivo or ex vivo tests on animals are at the heart of an intense and controversial debate on biomedical testing.

It is known that in the vascular tissue engineering industry, such as for example the tissue-engineered constructs industry, it is crucial to be able to produce a functional and preferably continuous endothelium (i.e. having a monolayer of confluent cells), mainly consisting of endothelial cells (ECs). The production of a functional and preferably continuous endothelium is a critical factor toward ensuring the adequate efficiency and safety of engineered vascular tissues or constructs, such as for example to prevent thrombosis and stenosis once said tissues or constructs are implanted.

The in vitro generation of engineered vascular construct or tissue having a functional and preferably continuous cell layer, preferably of endothelial cells, (for example a vascular endothelium) comprises, in short, the following steps:
seeding endothelial cells in the lumen of the scaffold and ensuing adhesion thereof, followed by
growth/proliferation and organisation of the endothelial cells as a function of the mechanical stimuli (such as the flow of a fluid) to which they are subjected, until one or more layers of functional and preferably continuous endothelial cells are obtained, and optionally,
characterisation of said one or more layers of cells being formed or formed.

In the seeding phase it is crucial to use a technique which allows to uniformly seed the cells, preferably endothelial cells, in the lumen of the scaffold, which allows a homogeneous adhesion of the cells to the scaffold and to generally increase the efficacy of the seeding. Just like the method used to stimulate and promote the growth of the cells, preferably endothelial cells, and the organisation thereof after seeding in order to obtain one or more functional and preferably continuous layers of cells, for example endothelial cells, is equally crucial.

Consequently, the need is felt to develop, for the production of engineered vascular tissues or constructs having a functional and preferably continuous cell layer (for example an endothelium), (i) a method for seeding cells, mainly endothelial cells, in the lumen of a scaffold, capable of ensuring the homogeneity and the uniform adhesion of endothelial cells, with the removal of air bubbles in the scaffold; and/or (ii) a method for stimulating (for example, a perfusion method) the growth/proliferation and organisation of cells, mainly endothelial cells, capable of ensuring the sterility and preservation of the absence of air bubbles in the production system.

There are various techniques used in the prior art for the production of engineered cardiovascular tissues or constructs and, in particular, for seeding and stimulating cell growth/proliferation and organisation; however, the results so far achieved are not entirely satisfactory.

Lastly, the need is felt to be able to determine the efficacy of the seeding method and of the method for stimulating the growth and cellular organisation selected by means of suitable characterisation methods capable of attesting the viability, morphology, functionality and organisation of the cells adhered in the lumen of the scaffold and the ensuing generation of a uniform, homogeneous, functional and preferably continuous cell layer (for example an endothelium).

Said characterisation methods must have the principle of not damaging the development of the cells and the adhesion thereof to the scaffold. In other words, said characterisation methods must not cause cellular alteration which could lead to the possible loss of the growing or formed cell layer (for example an endothelium).

Said characterisation methods, said seeding and adhesion method and said method for stimulating cell growth and organisation must be simple, fast, irrespective of the operator, highly reproducible, reliable and effective, so as to produce and characterise said engineered vascular constructs or tissues at laboratory and industrial level, in particular for GLP (Good Laboratory Practice)-certified tissue or construct engineering laboratories or industries.

Lastly, the need is felt to be able to develop and characterise in vitro engineered vascular tissues or constructs comprising a functional and preferably continuous cell layer (for example an endothelium) for performing advanced preclinical or clinical tests, so as to avoid using laboratory animals.

After a long and intense research and development activity with the aim of meeting the requirements described above, the Applicant developed a method for characterising a tissue-engineered construct that can be used for the in vitro testing of medicinal products for human or animal use, having the characteristics as claimed in the attached claims. In particular, said characterisation method allows to verify the viability, the morphology, the functionality, the distribution and/or other properties of the cells or cell layer adhered to the lumen of the scaffold of the tissue-engineered construct.

The methods of the present invention allow to overcome the limitations of the currently available methods and they offer a valid alternative to using animal models.

Preferred embodiments of the present invention will be clear from the detailed description that follows.

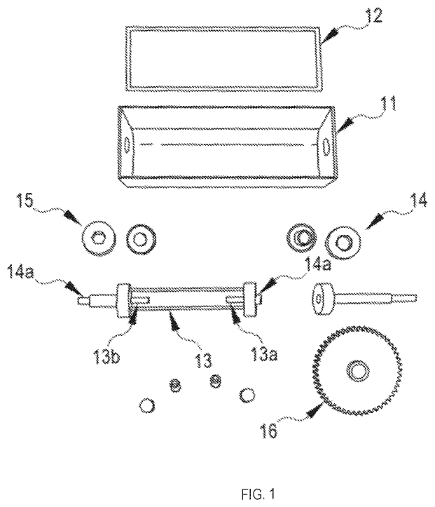

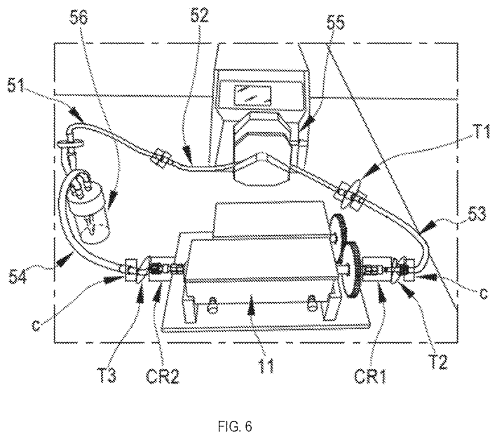

FIGS. 1-32 are described hereinafter in the present description.

In the context of the present invention, the expression tissue-engineered construct is used to indicate a scaffold having the lumen lined with at least one functional and preferably continuous layer of cells, preferably endothelial cells, that is to say a monolayer of confluent cells (for example a functional and continuous endothelium).

In the context of the present invention the terms engineered vascular tissue and tissue-engineered construct are used interchangeably.

In the context of the present invention the expression scaffold is used to indicate a biocompatible porous polymeric medium capable of promoting the cell adhesion and growth, endothelial cells in this case.

In the context of the present invention the expression functional endothelium is used to indicate an endothelium with physiological-like behaviour, wherein the endothelial cells are adjacent to each other, adhered to the scaffold and expressing markers typical of the endothelial cells, such as for example Von Willebrand factor (VWF), cluster of differentiation 31 (CD31), vascular cell adhesion molecule 1 (VCAM-1). Furthermore, the expression continuous endothelium is used to indicate an endothelium having a monolayer of confluent cells (for example at least at 90%).

In the context of the present invention the cells constituting an endothelium, preferably an endothelium of a vascular tissue, are defined as endothelial cells.

In the context of the present invention, the cell growth and maintenance fluid, specific for each type of cell, is defined as growth medium. For example, for HUVECs (Human Umbilical Vein Endothelial cells; Sigma Aldrich, code 200-05n) endothelial cells, the growth medium that can be used may be the Endothelial Growth Medium (abbreviated as EGM, Sigma Aldrich, code 211-500). EGM contains fetal bovine serum (2%), adenine (0.2 μg/ml), ammonium metavanadate (0.0006 μg/ml), amphotericin B (0.3 μg/ml), calcium chloride 2H₂0 (300 μg/ml), choline chloride (20 μg/ml), copper sulphate 5H₂0 (0.002 μg/ml), trioptic acid DL-6.8 (0.003 μg/ml), folinic acid (calcium) (0.6 μg/ml), heparin (4 μg/ml), hydrocortisone (2 μg/ml), L-aspartic acid (15 μg/ml), L-cysteine (30 μg/ml), L-tyrosine (20 μg/ml), manganese sulphate monohydrate (0.0002 μg/ml), ammonium molybdate 4H₂0 (0.004 μg/ml), nicotinamide (8 μg/ml), nickel chloride 6H₂0 (0.0001 μg/ml), penicillin (60 μg/ml), phenol red sodium salt (15 μg/ml), potassium chloride (300 μg/ml), putrescine dihydrochloride (0.0002 μg/ml), pyridoxine hydrochloride (3 μg/ml), sodium metasilicate 9H2O (3 μg/ml), sodium sulphate 7H₂O (200 μg/ml), sodium selenite (0.01 μg/ml), streptomycin sulphate (100 μg/ml), thiamine hydrochloride (4 μg/ml), and zinc sulphate 7H₂O (0.0003 μg/ml). A fresh growth medium is a sterile medium not used previously, directly supplied by the manufacturer. The expression hot growth medium is used to indicate a growth medium heated previously at a temperature comprised in the range between 20° C. and 45° C., preferably at 37° C.

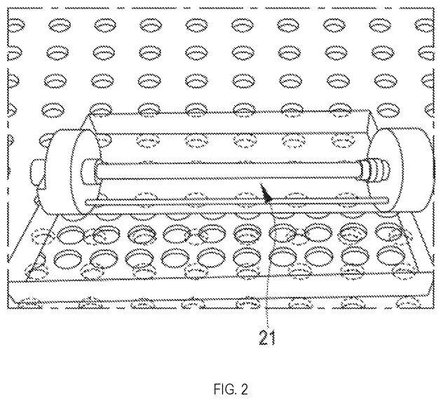

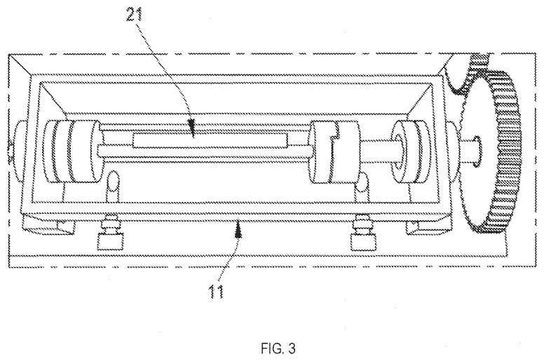

Forming an object of the present invention is a method for characterising (hereinafter, characterisation method of the present invention) a tissue-engineered construct, comprising a scaffold having at least one portion of the lumen lined with at least one functional cell layer, that can be used for the in vitro testing of medicinal products for human or animal use, said method comprising:
step I for preparing a scaffold (FIG. 2, 21) in a chamber of a bioreactor (FIG. 3, 11), to obtain a bioreactor (11)-scaffold (21) system, followed by
step II for applying a seeding method for seeding at least one portion of the lumen of said scaffold (21) with a cell culture and allowing the adhesion of said cells to the scaffold (21) to obtain a seeded bioreactor (11)-scaffold (21) system, followed by
step III for stimulating the growth and the organisation of said cells until at least one layer of functional cells is formed, followed by or concomitant with
step IV for characterising said adhered cells to the lumen of the scaffold (21) to verify the viability, morphology, functionality, distribution and/or other properties thereof known to the man skilled in the art.

Forming an object of the present invention is a method for characterising (hereinafter, characterisation method of the present invention) a tissue-engineered construct, comprising a scaffold having at least one portion of the lumen lined with at least one functional cell layer, that can be used for the in vitro testing of medicinal products for human or animal use, said method comprising the steps of:
step I. preparing a scaffold 21 in a chamber of a bioreactor 11, to obtain a bioreactor 11-scaffold 21 system, followed by
step II. applying a seeding method for seeding at least one portion of the lumen of said scaffold 21 with a cell culture and allowing the adhesion of said cells to the scaffold 21 to obtain a seeded bioreactor 11-scaffold 21 system, followed by
step III. stimulating the growth and the organisation of said seeded cells in the lumen of the scaffold 21 until at least one layer of functional cells adhered to at least one portion of the lumen of the scaffold 21 is formed, followed by or concomitant with
step IV. characterising said cells lining at least one portion of the lumen of the scaffold 21 to verify the viability, morphology, functionality, distribution and/or other properties thereof known to the man skilled in the art;

wherein step IV. of characterising said cells lining at least one portion of the lumen of the scaffold (21) comprises at least one non-destructive method to be applied to the cells during the step III. of forming at least one layer of functional cells, and/or at least one non-destructive method to be applied upon completing the step III. of forming at least one layer of functional cells.

According to an embodiment of the characterisation method according to the invention comprising steps I-IV (and optionally steps I.1, II.1-II.2, II.1-II.4, II.1-II.9, III.1-III.3, 2.1-2.11 and/or 3.1-3.13), the cells lining at least one portion of the lumen of said scaffold are endothelial cells selected from among the cells constituting an endothelium of a vascular tissue; preferably selected from among HAOECs (human aortic endothelial cells), HCAECs (human coronary artery endothelial cells), HMVECs (human dermal microvascular endothelial cells), and HUVECs (human umbilical vein endothelial cells).

According to an embodiment of the characterisation method according to the invention comprising steps I-IV (and optionally steps I.1, II.1-II.2, II.1-II.4, II.1-II.9, III.1-III.3, 2.1-2.11 and/or 3.1-3.13), the lumen of said scaffold is at least partly lined with at least one functional and continuous cell layer preferably a monolayer of functional and continuous endothelial cells having the confluent cells (i.e. functional and continuous endothelium).

According to an embodiment of the characterisation method according to the invention comprising steps I-IV (and optionally steps II.1-II.2, II.1-II.4, II.1-II.9, III.1-III.3, 2.1-2.11 and/or 3.1-3.13), said step I for preparing a scaffold comprises the step of:

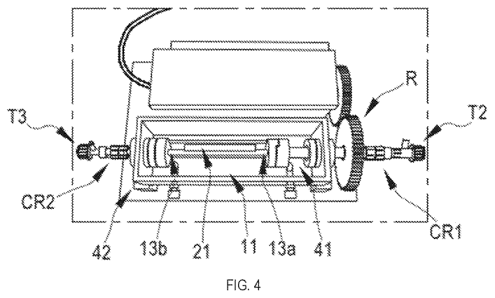

I.1: mounting the scaffold (21) in the bioreactor chamber (11), to obtain the bioreactor (11)-scaffold (21) system. Preferably, mounting the scaffold (21) on grips of a scaffold-holder (FIG. 1, 13, 13a, 13b) and housing said scaffold-holder (13, 13a, 13b) with the scaffold (21) in the bioreactor chamber (11). Applied at both ends of the bioreactor (upstream and downstream) are rotary connectors (FIG. 4; CR1, CR2) and T-shaped connectors (FIG. 4; T2, T3).

In the context of the present invention, the term bioreactor-scaffold system is used to indicate the bioreactor and scaffold assembly (FIG. 3; 11, 21), preferably a substantially tubular-shaped scaffold, which is housed and fixed into the bioreactor, for example to a scaffold-holder. The scaffold can be gripped by the grips of the scaffold-holder (which is internally hollow so as to allow the perfusion of the scaffold) with self-fastening strips, after having protected the scaffold with a sterilised teflon tape. The insertion of the scaffold-holder (13) into the bioreactor 11 occurs in a manner such that the inlet upstream of the bioreactor chamber coincides (FIG. 4; CR1, 41) with an end of the scaffold (FIG. 4; 13a) and the opening downstream of the bioreactor chamber (FIG. 4; CR2, 42) coincides with the other end of the scaffold (FIG. 4; 13b). In this manner, the scaffold (21) is perfectly coaxial with respect to the perfusion path generated by the scaffold-holder. The larger axis—according to which the scaffold mounted in the bioreactor—is oriented is defined as the longitudinal axis.

The scaffold (21) of the present invention is a polymeric scaffold of synthetic or natural origin and it consists of only one polymer or copolymers (set of polymers), such as for example electrospun silk fibroin or PGA/PLA (polyglycolic acid/polylactic acid) or PGA/PCL (polyglycolic acid/polycaprolactone) copolymers.

Preferably the scaffold of the present invention is made of substantially tubular-shaped electrospun silk fibroin.

According to an embodiment of the characterisation method according to the invention comprising steps I-IV (and optionally steps, I.1, III.1-III.3, 2.1-2.11 and/or 3.1-3.13), said seeding method comprised in step II, comprises the steps of:

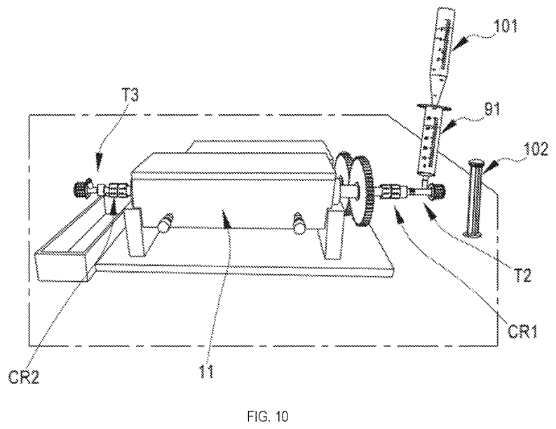

II.1: releasing said cell culture, preferably endothelial cells, in the form of a cell suspension comprising a fresh growth medium and cells, preferably endothelial cells, in a container (FIG. 10, 91) mounted on a T-shaped connector (FIG. 10, T2) arranged upstream of the bioreactor (11) by means of a rotary connector (FIG. 10, CR1); followed by

II.2: releasing said cell culture, preferably endothelial cells, in the inner lumen of the scaffold (21) present in the bioreactor (11) with a continuous flow so that the flow velocity allows said cell suspension to drip into the T-shaped connector (T2) without generating air bubbles and push the air bubbles present in the inner lumen of the scaffold (21) toward an opening of the T-shaped connector (FIG. 10, T3) arranged downstream of the bioreactor (11) allowing the outflow thereof.

Said container (FIG. 10, 91) can be the hollow portion of a syringe or the like.

Steps II.1 and II.2 of said seeding method reduce the risk of air bubbles coming into contact with the seeded cells, preferably endothelial cells, thus avoiding damage to the cells and allowing to obtain a monolayer of functional and preferably continuous cells (i.e. confluent cells) adhered to the lumen of the scaffold (for example a functional and preferably continuous endothelium).

According to an embodiment of the characterisation method according to the invention comprising steps I-IV (and optionally steps I.1, III.1-III.3, 2.1-2.11 and/or 3.1-3.13), wherein said seeding method comprised in step II comprises—besides steps II.1 and II.2—subsequently to step II.2, the steps of:

II.3: continuously rotating the scaffold (21) along the longitudinal axis thereof for a time interval comprised between 2 and 48 hours, preferably 24 hours, with a rotational speed comprised between 0.5 and 5 rpm, preferably between 1.5 and 2 rpm, more preferably for 24 hours at 1.5-2 rpm, so as to allow the adhesion of cells to the inner lumen of the scaffold (21) uniformly; followed by or concomitant with

II.4: incubating the scaffold (21) housed in the bioreactor (11) for a time interval comprised between 2 and 48 hours, preferably for 24 hours, at a temperature between 20 and 45° C., preferably at 37° C., in the presence of CO₂ at 1-10%, preferably at 5%; more preferably for 24 hours at 37° C. in presence of 5% of CO₂.

Preferably, step II.4 for incubating occurs with the scaffold (21) rotating according to step II.3.

Step II.3 for rotating improves the cell adhesion to the lumen of the scaffold and makes it uniform, it allows the scaffold to remain continuously wet by the growth medium present in the bioreactor chamber and it allows the through-flow of nutrients between the medium present in the chamber (outside the scaffold) and the cell suspension one seeded in the lumen of the scaffold.

According to an embodiment of the characterisation method according to the invention comprising steps I-IV (and optionally steps I.1, III.1-III.3, 2.1-2.11 and/or 3.1-3.13), said seeding method according to steps II.1-II.4 in detail comprises steps II.1-II.9 illustrated below, carried out in sequence and under sterile conditions.

Step I.1 is followed by step II.5: injecting the fresh growth medium into the lumen of said scaffold (21) fixed on said scaffold-holder (13) arranged in the bioreactor chamber (11) (i.e. preconditioning of the scaffold). Step II.5 is followed by step II.6: adding said fresh growth medium into the bioreactor chamber (11) where said scaffold-holder (13, 13a, 13b) with the scaffold (21) is present injected with said growth medium. Step II.6 is followed by step II.7: leaving for a time interval comprised between 1 hour and 18 hours at a temperature comprised between 20° C. and 30° C., preferably 25° C., said growth medium in the inner lumen of the scaffold (21) and in the bioreactor chamber (11) where said scaffold-holder (13) with the scaffold (21) is present injected with said growth medium.

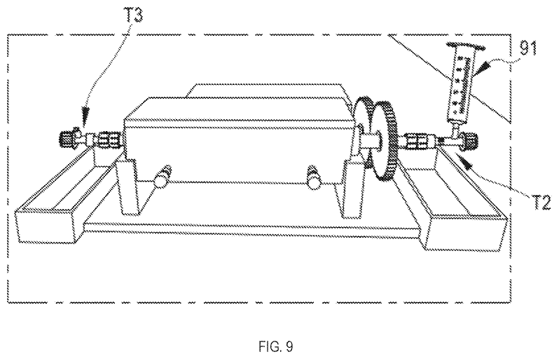

In detail, the scaffold is preconditioned using a syringe with luer-lock connector which is coupled to one of the two ends of the bioreactor by means of a T-shaped connector (FIG. 9; T2). Subsequently, the open ends of the connectors arranged upstream and downstream of the bioreactor are closed using caps so as to avoid the emptying of the lumen of the scaffold. Furthermore, the fresh growth medium is inserted into the bioreactor chamber until the scaffold housed therein is fully covered. In this manner, the scaffold housed in the bioreactor chamber is preconditioned, preferably for about 1 hour at about 25° C., using a fresh growth medium both internally (in the lumen) and externally.

Step II.7 is followed by step II.8: clearing the inside of the lumen of the scaffold (21) and the bioreactor chamber (11) from the growth medium, preferably using a sterile pipette. The growth medium residues present in the connectors (rotated and T-shaped) arranged downstream and upstream of the bioreactor are eliminated with a vacuum using a pipette, without making the scaffold collapse.

Step II.8 is followed by step II.1, previously described, for releasing said cell culture, preferably endothelial cell culture, in said container (91) according to step II.1, preferably said container (91) is a syringe.

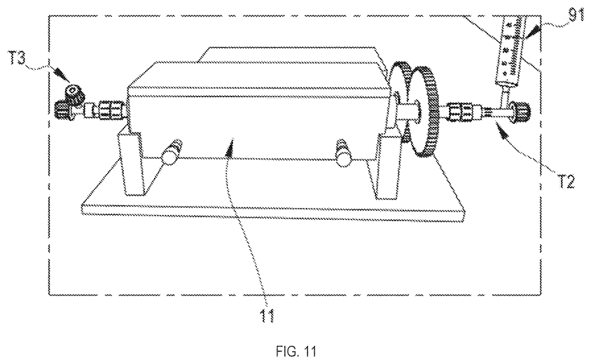

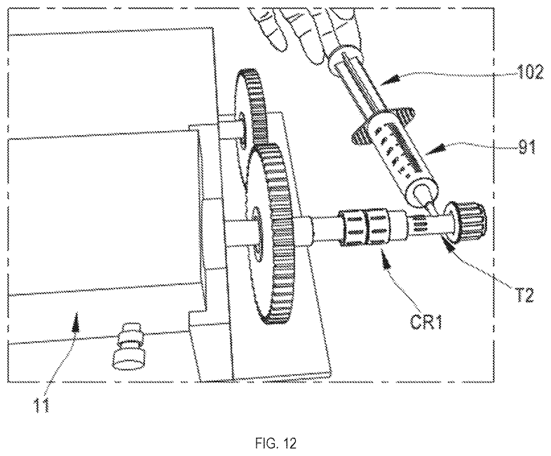

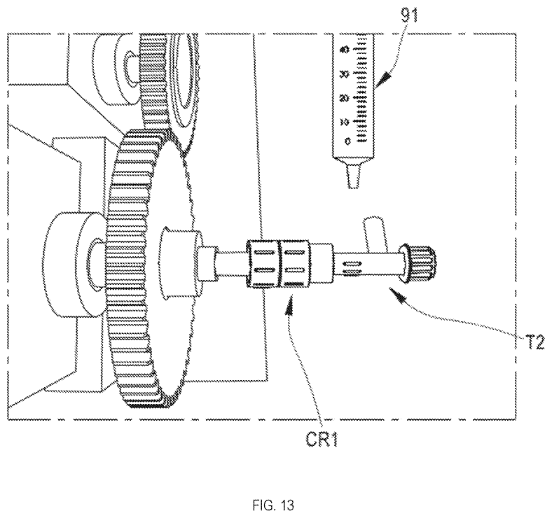

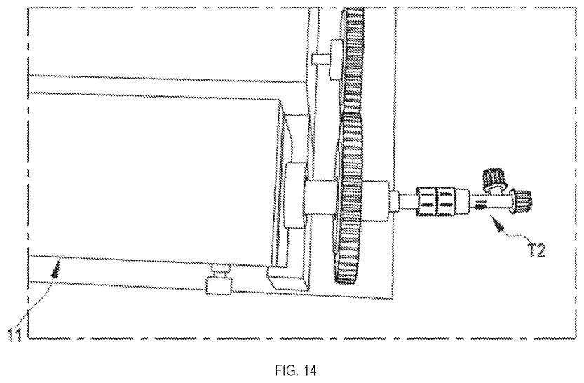

Step II.1 is followed by step II.2, previously described, for releasing said cell culture, preferably endothelial cells, in the inner lumen of the scaffold (21) present in the bioreactor (11) with a continuous flow. In detail, the T-shaped connectors—arranged upstream (FIG. 9, T2) and downstream (FIG. 9; T3) of the ends of the bioreactor, with the scaffold therein mounted on the grips—are directed with the upper opening upwards (at 90° with respect to the plane in which the bioreactor-scaffold system lies). Subsequently, the lateral opening of the T-shaped connectors arranged downstream (T3) and upstream (T2) of the bioreactor is capped. Mounted on the opening facing upwards of the T-shaped connector (FIG. 9; T2) arranged upstream of the bioreactor is a container, preferably a syringe (FIG. 9; 91) with a luer-lock connector with capacity for example of 5 ml without the plunger thereof. The opening of the T-shaped connector (FIG. 9; T3) arranged downstream of the bioreactor remains open instead (FIG. 9). Using a pipette, preferably a sterile plastic pipette with capacity of for example 25 ml (FIG. 10; 101), a cell suspension consisting of fresh growth medium and endothelial cells (e.g. HUVECs) is drawn from a container in which it was prepared. Subsequently, the drawn cell suspension is released into the container or into the syringe (FIG. 10; 91) mounted on the T-shaped connector element (FIG. 10; T2) upstream of the bioreactor through the element (FIG. 10; CR1). The cell suspension must be released, using the pipette (FIG. 10; 101), with capacity of for example 25 ml, with a continuous flow so that the flow velocity allows the cell suspension to drip into the T-shaped connector (FIG. 10; T2) without generating air bubbles and to push possible air bubbles present in the scaffold toward the opening of the T-shaped connector T3 arranged downstream (FIG. 10; T3) of the bioreactor 11 and, hence to flow out (FIG. 10). When the cell suspension—loaded using a syringe—reaches the open end of the T-shaped connector T3 arranged downstream of the bioreactor without possible air bubbles, the opening of the T-shaped connector T2 arranged upstream of the bioreactor is closed using a cap (FIG. 11). Subsequently, the syringe (91) with the cell suspension residue is rotated by about 90° with respect to the plane on which it lies (FIG. 12); in this position the plunger of the syringe (102) is re-inserted at the open end of the syringe (FIG. 12) by inserting the insulating black part only so as not to create pressure inside the scaffold. Subsequently, the syringe (91) can be unscrewed from the T-shaped connector T2 upstream of the bioreactor without forming air bubbles (FIG. 13) and the end of the connector is closed using a cap (FIG. 14).

Step II.2 is followed by step II.9: adding hot fresh growth medium (as previously defined) into the bioreactor chamber (11) where said scaffold-holder (13) is present with the seeded scaffold (21) containing said cell suspension in the lumen, until the scaffold is half-immersed into the growth medium.

Step II.9 is followed by step II.3, previously described, for continuously rotating the scaffold (21) according to step II.3.

In detail, a continuous rotation is then applied along the longitudinal axis of the scaffold, for example with a rotation speed comprised between 1.5 and 2 rpm, for 24 hours. The rotation allows the uniform cell adhesion to the lumen of the scaffold, allows the scaffold to remain continuously wet by the growth medium present in the bioreactor chamber and it allows the through-flow of nutrients between the medium present in the chamber (outside the scaffold) and the cell suspension one seeded in the lumen of the scaffold.

Step II.3 is followed by step II.4, previously described, for incubating the scaffold (21) housed in the bioreactor chamber (under rotation), preferably for 24 hours at 37° C. with 5% of CO2.

Advantageously, the presence of the seeding method according to steps II.1-II.2 or steps II.1-II.4 or steps II.1-II.9 in the characterisation method according to steps I-IV (and optionally steps I.1, III.1-III.3, 2.1-2.11 and/or 3.1-3.13) allows to operate under sterile conditions and to seed the cells eliminating both the air bubbles present in the bioreactor-scaffold system and those which are formed during seeding, thus avoiding to damage the cells. This allows the production of an engineered vascular tissue or construct having a scaffold having at least one portion of the lumen lined with a functional and preferably continuous layer of cells (i.e. having a monolayer of confluent cells), such as a continuous and functional endothelium. Basically, besides being quick, standardised and reproducible, each step of the present seeding method optimises the cost and the operating time.

According to an embodiment of the characterisation method according to the invention comprising steps I-IV (and optionally steps I.1, II.1-II.2, II.1-II.4, II.1-II.9, 2.1-2.11 and/or 3.1-3.13), said step III for stimulating the growth and the organisation of said cells, preferably endothelial cells, comprises a step of applying a perfusion method with a hot fresh growth medium having a temperature comprised in the range between 20° C. and 45° C., preferably 37° C., of the cells present in the lumen of said seeded scaffold (21), wherein said perfusion method comprises steps III.1-III.3 described below.

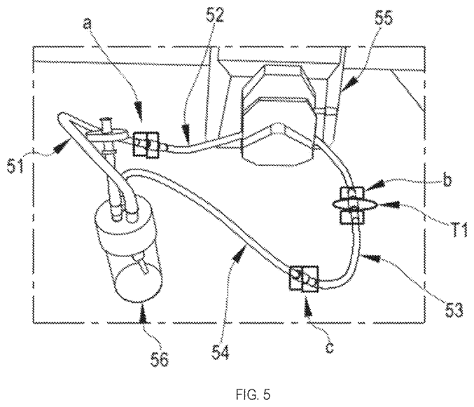

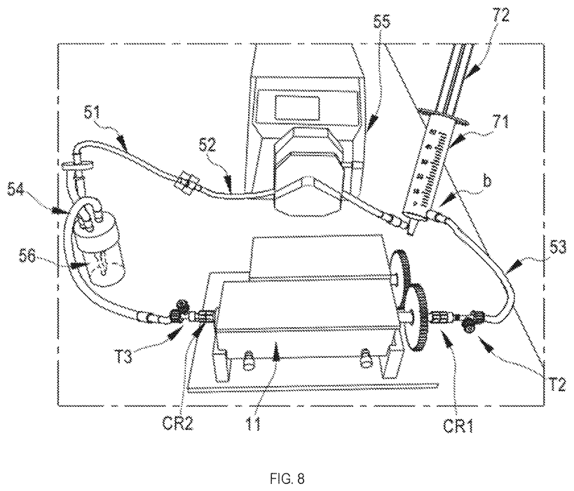

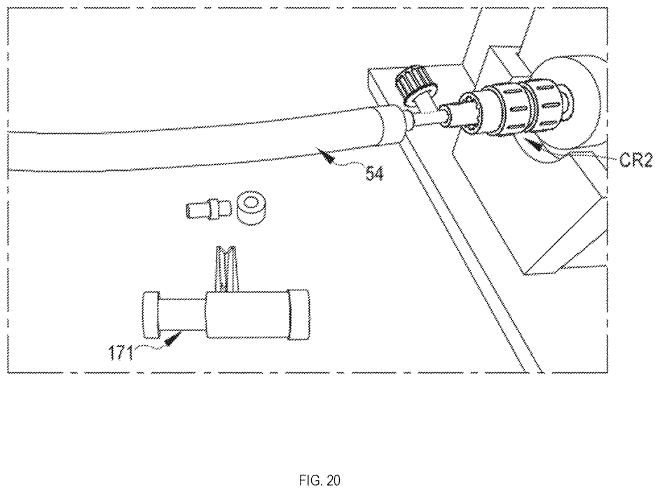

Step III.1: connecting, in variable sequence, an element for removing air bubbles (FIG. 8, 71-72 or FIG. 22, BT) and said seeded bioreactor (11)-scaffold (21) system to a perfusion circuit (FIG. 5, 51-56), wherein said element for removing air bubbles is inserted upstream of the seeded bioreactor (11)-scaffold (21) system; the perfusion circuit (FIG. 5, 51-56) comprises a reservoir (FIG. 5, 56) containing a growth medium. Step III.1 is followed or preceded by step III.2: filling at least one part of said element for removing air bubbles (FIG. 8, 71-72 or FIG. 22 BT) with said hot fresh growth medium, wherein said element for removing air bubbles (FIG. 8, 71-72 or FIG. 22 BT) comprises a chamber, a cap for closing said chamber, an access with inflow function (211) and an access with outflow function (212), wherein said chamber has a volume and wherein a first part of said volume is filled with said fresh growth medium and wherein a second part of said volume is filled with air, said second part of said volume having the function of trapping the air bubbles present in said fresh growth medium which flows through said access with inflow function (211) and said access with outflow function (212).

Preferably, said element for removing air bubbles (FIG. 8, 71-72, or FIG. 22, BT) is a bubble trap or the like. Step III.1 and III.2 are followed by step III.3: allowing the perfusion of the seeded scaffold (21) with said hot fresh growth medium, preferably by means of a peristaltic pump.

Steps III.1-III.3, comprised in step III for stimulating the growth and the organisation of the cells until at least one layer of functional cells is formed, comprised in the characterisation method according to the invention comprising steps I-IV (and optionally steps I.1, II.1-II.2, II.1-II.4, II.1-II.9), can be carried out according to a first embodiment comprising steps 2.1-2.11 (shown in FIGS. 5-8, 18-21) or, alternatively, according to a second embodiment comprising steps 3.1-3.13 (shown in FIGS. 22-27), described below. The steps of said first embodiment (steps 2.1-2.11) and said second embodiment (steps 3.1-3.13) are carried out in sequence and under sterile conditions.

Said steps 2.1-2.11 or 3.1-3.13 are subsequent to step II carried out according to steps II.1-II.2 or II.1-II.4 or II.1-II.9.

Said first embodiment (steps 2.1-2.11), in short, firstly provides for the connection of the perfusion circuit to the seeded bioreactor-scaffold system, then the filling of the element for removing air bubbles with a growth medium and subsequently the insertion of the element for removing air bubbles into the perfusion circuit previously connected to the seeded bioreactor-scaffold system.

2.1 Place the tube or under-pump (FIG. 5; 52) of the closed perfusion circuit under the head of the peristaltic pump (FIG. 5; 55), which—upon activation—generates a peristaltic force capable of suctioning fluids, hot fresh growth medium in this case. The closed perfusion circuit is filled due to the suctioning, by the tube (FIG. 5; 51) of the perfusion circuit connected to the reservoir (FIG. 5; 56), of the hot fresh growth medium which is previously poured into the reservoir once closed. Fill all the tubes of the perfusion circuit with the hot fresh growth medium until the fresh growth medium returns to the reservoir through the tube 54 (FIG. 5; 54) of the closed perfusion circuit. Place the bioreactor-scaffold system under the same sterility conditions as the perfusion circuit.

2.2 Open the upper and lateral ends of the T-shaped connector T2 arranged upstream of the bioreactor.

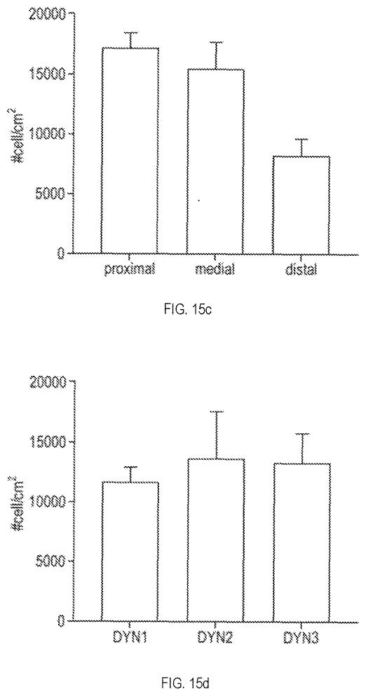

2.3 Upon removing the caps from the upper and lateral ends of the T-shaped connector T2 arranged upstream of the bioreactor, the possible creation of air bubbles is compensated by manually adding (preferably using a pasteur pipette) having the same volume as the hot fresh growth medium (FIG. 16).

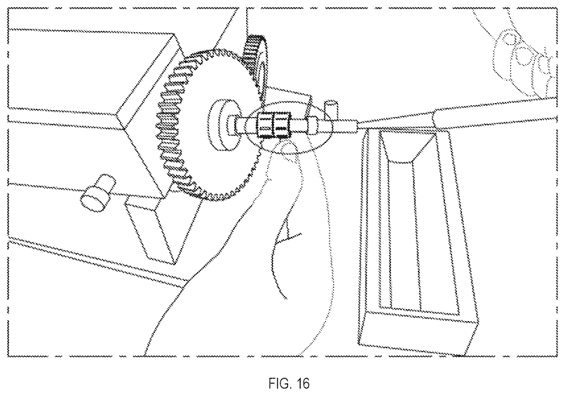

2.4 Occlude the tube 54 of the closed perfusion circuit, preferably using a clamp (FIG. 17; 171) in a position proximal to the connector between the tube 54 and the tube 53 of the perfusion circuit (FIG. 17). Be careful to keep the head of the pump closed so as to prevent the emptying of the tube 53 of the closed perfusion circuit.

2.5 Hold the tube 53 of the closed perfusion circuit in vertical position, unscrew the connector (FIG. 5, C) arranged between the tube 53 and the tube 54 of the perfusion circuit and preferably close the tube 54 of the perfusion circuit with a cap.

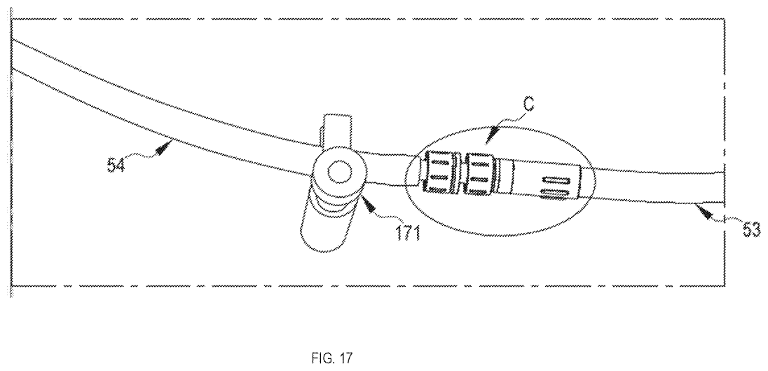

2.6 Screw the tube 53 of the perfusion circuit to the open lateral end of the T-shaped connector T2 upstream of the bioreactor at a lateral access thereof. The connector upstream of the bioreactor must always be kept in vertical position (FIG. 18).

2.7 Open the T-shaped connector T3 downstream of the bioreactor by unscrewing the cap of the lateral opening.

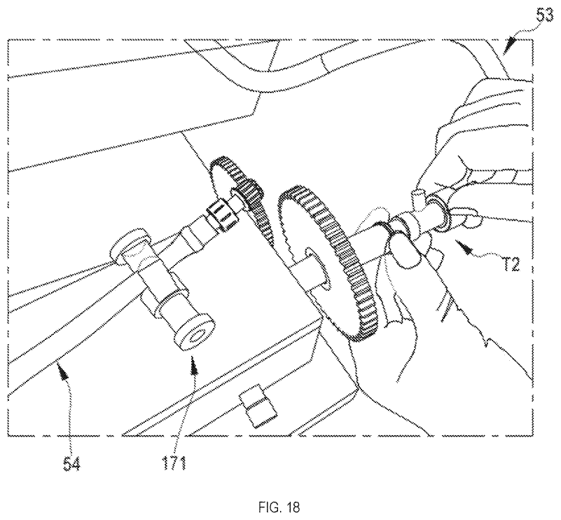

2.8 Remove the cap from the connector of the tube 54 of the perfusion circuit (FIG. 19A) and screw it onto the lateral opening of the T-shaped connector downstream of the bioreactor (FIG. 19B).

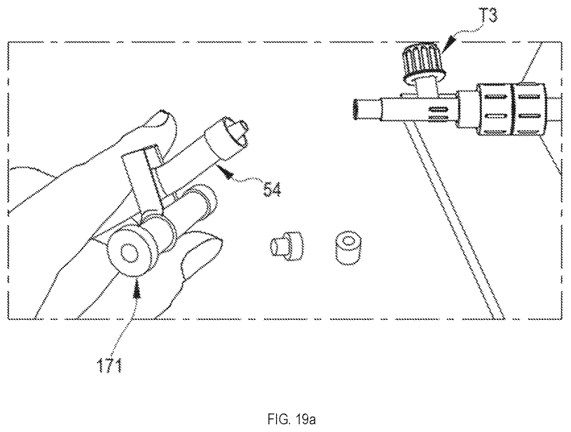

2.9 Remove the clamp 171 which occludes the tube 54 of the perfusion circuit (FIG. 20).

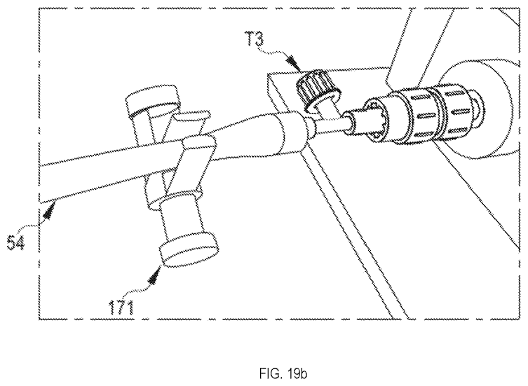

2.10 Fill the element for removing air bubbles (FIG. 21, 71-72), defined with the technical expression of bubble trap (BT) in the context of the present invention. The bubble trap consists of an element represented by a chamber closed using a cap and having two accesses having inflow and outflow function. The bubble trap chamber contains a volume of liquid (hot fresh growth medium in this specific case) and a volume of air that traps possible air bubbles present in the perfusion liquid which flows through the two accesses of the bubble trap chamber. Fill the bubble trap with hot fresh growth medium so as to leave a given air volume and close the chamber as well as its two accesses using the respective caps.

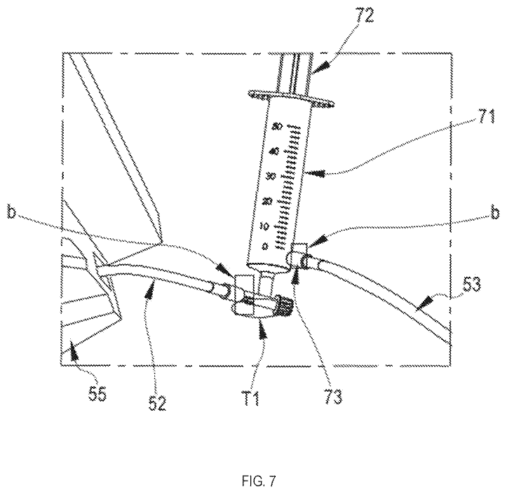

2.11 Connect the bubble trap to the perfusion circuit previously connected to the bioreactor-scaffold system as follows (FIG. 7):

a. close the tube 53 of the perfusion circuit using a clamp arranged proximally to the connector which connects the tube 53 to the lateral end of the T-shaped connector T1 (arranged between the tube 53 and the tube 52 of the perfusion circuit) and unscrew it (FIG. 7; 52).

b. preferably, close the tube 53 of the perfusion circuit using a cap. Such operation prevents the emptying of the tube 53 of the perfusion circuit.

c. cap the lateral end of the T-shaped connector T1 (arranged between the tube 53 and the tube 52 of the perfusion circuit) and open the upper end thereof.

d. open the inflow access of the bubble trap chamber and connect it to the access of the upper end and arranged vertically with respect to the T-shaped connector T1.

e. open the outflow access of the bubble trap and connect it to the tube 53 of the perfusion circuit, being careful not to twist the tube at all.

f. keep the bubble trap in vertical position.

g. remove the clamp 71 from the tube 53 of the perfusion circuit just connected to the bubble trap chamber.

Start the pump and open the cap of the upper end of the T-shaped connector T2 upstream of the bioreactor so as to eliminate possible air bubbles formed in the tube 53 during the process preventing them from reaching the scaffold. The peristaltic force applied by the pump to the assembled system, consisting of the perfusion circuit and the bioreactor-scaffold system will allow the perfusion of the scaffold.

h. after such verification, close the T-shaped connector T2 upstream of the bioreactor using the cap thereof.

Said second embodiment (steps 3.1-3.13), in short, firstly provides for the connection of the element for removing bubbles to the perfusion circuit, subsequently the filling of the element for removing air bubbles with a growth medium and, lastly, the insertion of the seeded bioreactor-scaffold system into the perfusion circuit previously connected to the element for removing air bubbles.

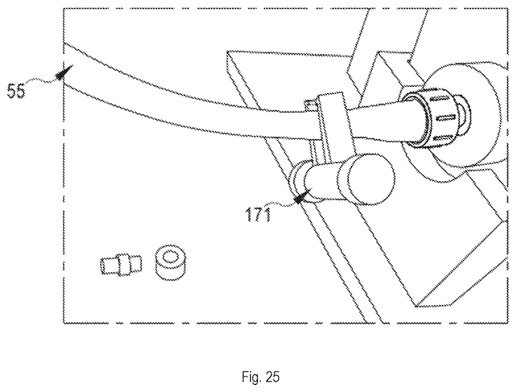

3.1 Connect—under sterile conditions—the tubes of the perfusion circuit (FIG. 22; 51; 52; 53; 54; 55), the element for removing air bubbles (FIG. 22; BT), defined in the context of the present invention with the technical expression bubble trap, and the reservoir (FIG. 22; 56). The element for removing the air bubbles or bubble trap (BT) consists of an element represented by a closed chamber, preferably made of glass, using a cap and having two asymmetric accesses: the access with the tap and connecting nozzle serves as an inflow (FIG. 27, 211) while access with the connecting nozzle only serves as an outflow (FIG. 27, 221). The bubble trap chamber contains a volume of liquid (hot fresh growth medium in this specific case) and a volume of air that traps possible air bubbles present in the perfusion liquid which flows through the two accesses of the bubble trap chamber.

3.2 Place the under-pump (FIG. 22; 52) of the closed perfusion circuit under the head of the peristaltic pump (FIG. 22; 57), which—upon activation—generates a peristaltic force capable of suctioning fluids, hot fresh growth medium in this case. The closed perfusion circuit is filled due to the suctioning, by the tube (FIG. 22; 51) of the perfusion circuit connected to the reservoir (FIG. 22; 56), of the hot fresh growth medium which is previously poured into the reservoir (FIG. 22; 56) in turn closed. Fill all the tubes of the perfusion circuit, the bubble trap (FIG. 22, BT) and the reservoir (FIG. 22; 56) with a perfusion liquid, such as a hot fresh growth medium (as defined above), until the hot fresh growth medium returns to the reservoir through the tube 55 (FIG. 22; 55) of the closed perfusion circuit. The BT and the reservoir are filled so as to leave a given air volume. In particular, the bubble trap (FIG. 22, BT) is filled so that said bubble trap chamber has a first part of the volume thereof filled with said fresh growth medium and a second part of the volume thereof filled with air, said second part of said volume having the function of trapping the air bubbles present in the perfusion liquid (hot fresh growth medium) which flows through said access serving as an inflow (211) and said access serving as an outflow (212) of the bubble trap (BT) (FIG. 22).

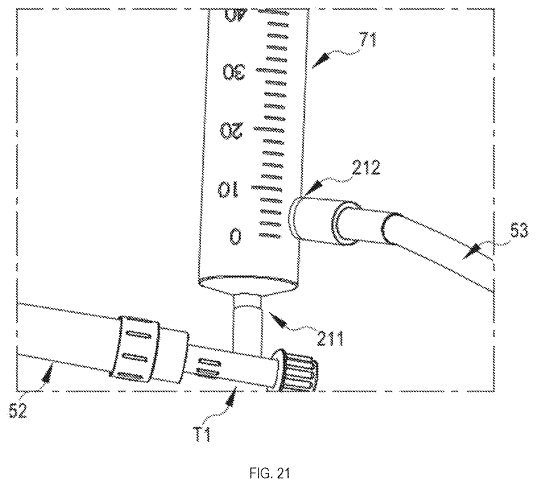

3.3 Occlude the tube 54 (FIG. 23), preferably using a clamp (FIG. 23; 172) in a position proximal to the BT and move the tap of the BT to the closing position (in perpendicular position with respect to the tubes of the perfusion circuit).

3.4 Place the bioreactor-scaffold system under the same sterile conditions as the perfusion circuit.

3.5 Occlude the tube 55 (FIG. 23), preferably using a clamp (FIG. 23; 171; FIG. 17; 171) in a position proximal to the connection C with the tube 54 (FIG. 23; C). Open the upper and lateral ends of the T-shaped connector T2 arranged upstream of the bioreactor (FIG. 4; T2).

3.6 Upon removing the caps from the upper and lateral ends of the T-shaped connector T2 (FIG. 4) arranged upstream of the bioreactor, the possible creation of air bubbles is compensated by manually adding (preferably using a pasteur pipette) having the same volume as the hot fresh growth medium (FIG. 16).

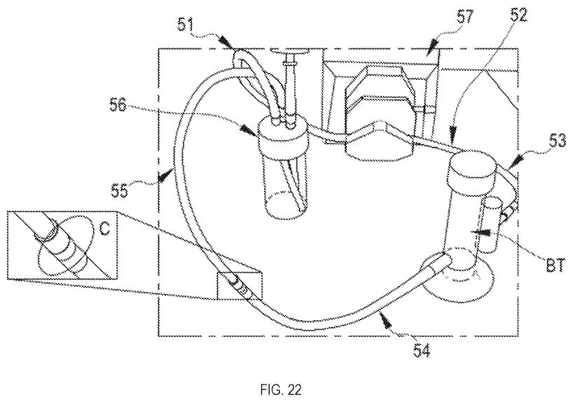

3.7 Hold the tube 54 (FIG. 23) of the closed perfusion circuit in vertical position, unscrew the connector arranged between the tube 54 (FIG. 23) and the tube 55 (FIG. 23) of the perfusion circuit and preferably cap the tube 55 of the perfusion circuit using a cap (FIG. 24).

3.8 Screw the tube 54 (FIG. 27) of the perfusion circuit to the open lateral end of the T-shaped connector T2 (FIG. 27) upstream of the bioreactor at a lateral access thereof. The connector upstream of the bioreactor must always be kept in vertical position (FIG. 18 or 27).

3.9 Open the T-shaped connector T3 (FIG. 4) downstream of the bioreactor by unscrewing the cap of the lateral opening.

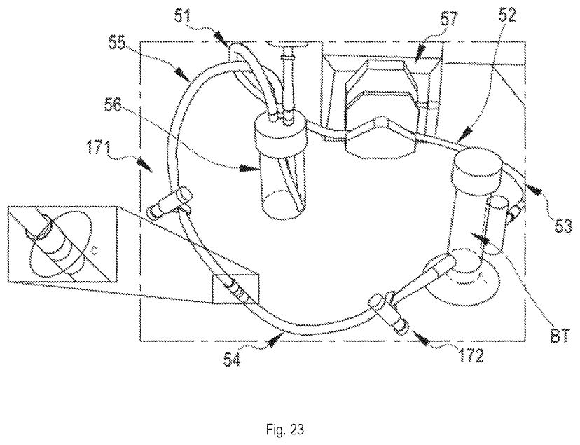

3.10 Unscrew the rotary connector T3 together with the T-shaped connector CR2 (FIG. 4), holding the toothed wheel R (FIG. 4) locked and screw the connector of the tube 55 (FIG. 25) on the lateral opening of the scaffold-holder 14a (FIG. 1) (FIG. 25).

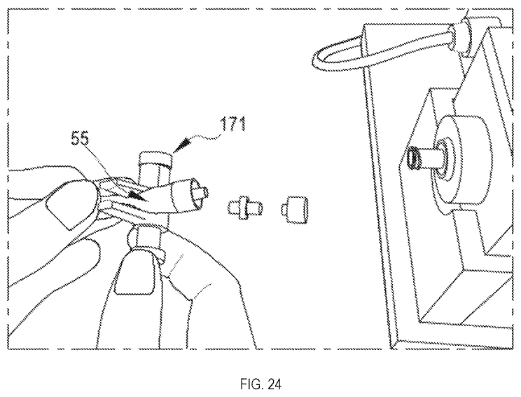

3.11 Remove the clamp 171 which occludes the tube 55 of the perfusion circuit (FIG. 26).

3.12 Place the seeded bioreactor-scaffold system connected to the perfusion circuit at about 37° C. and at about 5% of CO₂, place the under-pump 52 (FIG. 27) in free position, open the tap of the bubble trap BT (FIG. 27) by positioning it parallel to the tubes of the perfusion circuit, remove the clamp 172 (FIG. 27).

3.13 Switch the pump ON (FIG. 27, 57). The peristaltic force applied by the pump to the assembled system, consisting of the perfusion circuit, the element for removing air bubbles and the bioreactor-scaffold system will allow the perfusion of the scaffold (step III.3).

In the context of the present invention, the perfusion circuit (FIG. 5 and FIG. 22) is defined as an assembly of: tubes (FIG. 5; 51-54 or FIG. 22; 51-55), a reservoir (FIG. 5 or FIG. 22; 56) and a peristaltic pump (FIG. 5; 55 or FIG. 22; 57). Said tubes are made of biocompatible material and they are connected to each other so as to allow the perfusion of the scaffold (FIG. 21 and FIG. 27), preferably substantially tubular-shaped, housed in the bioreactor 11, by means of the peristaltic pump (FIG. 5; 55, FIG. 22; 57) (in such case, Masterflex®, L/S Digital Dispensing Pump Drives 07551-20, Cole-Parmer) with the Easy-Load II 77200-62 (Masterflex, Cole-Parmer) head.

With reference to the second embodiment of the perfusion method (steps 3.1-3.13) described above and illustrated in FIG. 27, the perfusion circuit mainly consists of five tubes with an inner diameter of 3/16″: a first tube 51 for suctioning from the reservoir, a second under-pump tube 52, a third tube 53 which connects the circuit to the bubble trap BT, a fourth tube 54 which connects the BT to the T-shaped connector T2 upstream of the bioreactor-scaffold system, a fifth tube 55 for return to the reservoir 56 connected to the T-shaped connector T3 downstream of the bioreactor-scaffold system.

With reference to the first embodiment of the perfusion method described above (steps 2.1-2.11) and represented in FIG. 5, the tube 51 is connected to the under-pump tube 52, the under-pump tube 52 to the BT, the BT to the tube 53, the 53 to the T-shaped connector T2 upstream of the bioreactor-scaffold system, the 54 to the T-shaped connector T3 downstream of the bioreactor-scaffold system and to the reservoir 56.

The reservoir (FIG. 5 or FIG. 22, 56) is the element containing the hot fresh growth medium (for example Endothelial Growth Medium EGM, Sigma Aldrich) from which the tube 51 (FIG. 5 or FIG. 22) suctions and to which the tube 54 (FIG. 5) or 55 (FIG. 22) returns, keeping the entire circuit and the bioreactor-seeded scaffold system in a closed system. The reservoir (FIG. 5 or FIG. 22, 56) is under atmospheric pressure, due to a 0.22 μm filter present on the cap of the reservoir, which guarantees the sterility of the air.

Advantageously, the perfusion method described herein (steps III.1-III.3), both in the first embodiment (steps 2.1-2.11) and in the second embodiment (steps 3.1-3.13), allows to connect a perfusion circuit and an element for removing air bubbles to the seeded bioreactor-scaffold system avoiding the creation of air bubbles and preventing the bubble, if formed, from reaching the scaffold seeded with cells, preferably endothelial cells, of the bioreactor-scaffold system. Furthermore, the air bubbles possibly already present in the perfusion circuit do not reach the scaffold due to the presence of the element for removing air bubbles (FIG. 21, 71-72 or FIG. 27, BT) in the circuit.

Thus, the presence in the characterisation method comprising steps I-IV (and, optionally, steps I.1, II.1-II.2, II.1-II.4 or II.1-II.9) of steps III.1-III.3, both in the first embodiment (steps 2.1-2.11) and in the second embodiment (steps 3.1-3.13), ensures—with greater safety with respect to the application of steps III.1-II.2, II.1-II.4 or II.1-II.9 only,—the complete absence of air bubbles in the lumen of the scaffold and the production of an engineered vascular tissue or construct having a scaffold having at least a portion of the lumen lined with a functional and preferably continuous layer of cells (i.e. having a monolayer of confluent cells), such as a continuous and functional endothelium.

Furthermore, during the step of connecting the seeded bioreactor-scaffold system and the perfusion circuit of the present perfusion method (step III.1), no changes occur in the state of cell adhesion, a crucial factor for allowing the growth of the vascular cell layer.

Step IV. for characterising said cells adhered to the lumen of the scaffold included in the tissue-engineered construct obtained by means of steps I-III (and, optionally, steps I.1, II.1-II.2, II.1-II.4, II.1-II.9, 11.1-111.3, 2.1-2.11 and/or 3.1-3.13), has the purpose of verifying and attesting the efficacy of said steps I-III (and optionally, of steps I.1, II.1-II.2, II.1-II.4, II.1-II.9, II.1-III.3, 2.1-2.11 and/or 3.1-3.13) and, in particular, of the seeding method and of the perfusion method used and, consequently, verifying and attesting the formation of a scaffold having at least one portion of the lumen lined with at least one layer of functional cells, preferably a layer of functional endothelial cells (functional endothelium); more preferably a layer of functional and continuous endothelial cells having confluent cells (functional and continuous endothelium).

Said characterising step IV. comprises analysing the viability, morphology, functionality, distribution and/or other suitable characteristics of said cells adhered to the scaffold by means of suitable analyses described below or known to the man skilled in the art.

These analyses must not damage the development of the cells and the adhesion thereof to the scaffold. As a matter of fact, the cells adhered to the lumen of the scaffold must preserve their morphology, viability and functionality in order to give rise to a functional, homogeneous and preferably continuous vascular endothelium (with confluent cells). Hence, it is important to avoid any cell alteration during the characterisation step IV which could lead to the possible loss of the growing or formed vascular layer. The analyses that can be performed can be distinguished into non-destructive analyses and destructive analyses.

Advantageously, the step IV. for characterising said cells, preferably endothelial cells, adhered to the scaffold comprises at least one non-destructive method to be applied to the cells during the step of forming at least one layer of functional cells and/or, alternatively, at least one non-destructive or destructive method to be applied upon completing the step of forming at least one layer of functional and preferably continuous layer of cells.

Preferably, said non-destructive method is an assay of metabolic reaction of cells to a reagent; whereas said destructive method is selected from among a DNA quantification assay or a colorimetric assay with DAPI (4′,6-diamidino-2-phenylindole, IUPAC name 2-(4-amidinophenyl)-1H-indole-6-carboxamidine, CASS 47165-04-8) or rhodamine-phalloidin, phalloidin CASS No 17466-45-4) or haematoxylin (IUPAC name 7,11b-dihydroindeno[2,1-c]chromene-3,4,6a,9,10(6H)-pentol, CASS No, 517-28-2) or eosin Y, IUPAC name 2-(2,4,5,7-tetrabromo-6-oxido-3-oxo-3H-xanthen-9-yl)benzoate in deprotonated form, CASS No 17372-87-1). The assays with destructive method are carried out on samples of said functional cell layer or on samples of said scaffold having the lumen lined with at least one functional cell layer, such as an endothelialized scaffold.

The viability of the cells adhered to the scaffold according to steps I-III of the characterisation method subject of the present invention is evaluated, for example, by means of an assay of metabolic reaction of the cells and/or by means of the quantification of the DNA present in the sample.

Said metabolic reaction assay for the evaluation of cell viability uses as reagent, for example, resazurin (trade name Alamar Blue, IUPAC name 7-hydroxy-10-oxidophenoxazin-10-ium-3-one, CAS 550-82-3) or other indicators known to the man skilled in the art. Such assay consists of a metabolic reaction that allows to quantify cell viability due to the oxidation-reduction of the indicator; for example, resazurin is reduced to resorufin, a pink fluorescent compound, in the presence of reducing atmosphere of a vital cell. Said metabolic reaction assay is a non-destructive method applicable to cells during the step of forming the layer of functional cells on the scaffold without jeopardising cell growth. Such metabolic reaction assay provides an arbitrary unit of fluorescence (abbreviated as A.U.) which is an indicator of cell viability but not of the cell count.

The viability of the cells can also be analysed by the assay for quantifying the genomic DNA present in the cells adhered to the scaffold, carried out on the samples used for the metabolic reaction assay. The DNA quantification assay provides a value of live and functional cells present in the sample and it allows to make the relative value of A.U., obtained with the metabolic reaction assay, an absolute value.

Such assay is based on the binding of Picogreen® (fluorescent compound) to the double strand of DNA and thus on the fluorescence emission of DNA into the visible sample at a suitable wavelength of the spectrophotometer. In particular, the genomic DNA is extracted from the cells adhered to the scaffold through lysis and it is subsequently quantified using Quant-iT™ PicoGreen™ dsDNA Assay (P7589, Invitrogen, Molecular Probes) where the fluorescent stain of the nucleic acids (PicoGreen®) allows to determine the concentration of genomic DNA in solution through a standard reference curve. This assay is a destructive method, therefore it can be used only when the step of forming the functional and preferably continuous layer of cells (e.g., functional and continuous endothelium) adhered to the scaffold has been completed, for the verification of the result.

The morphology of the cells adhered to the scaffold according to steps I-III of the characterisation method subject of the present invention can be verified, for example by means of the colorimetric assay with DAPI or the colorimetric assay with rhodamine-phalloidin or the colorimetric assay with haematoxylin or the colorimetric assay with eosin or any other assay known to the man skilled in the art Morphology characterisation assays are destructive assays.

The two assays with DAPI and haematoxylin allow to evaluate the cell morphology by staining the core and by observing under an optical fluorescence microscope or a confocal microscope; whereas the two assays with rhodamine-phalloidin and eosin allow to evaluate the cell morphology by staining the cytoplasm.

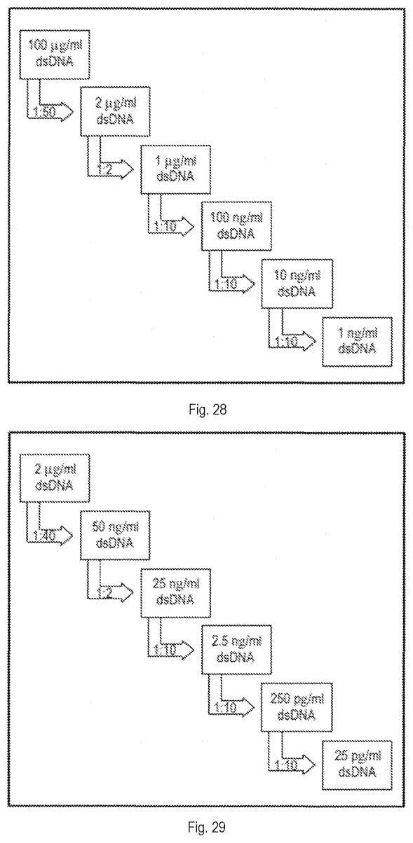

DAPI staining is based on the principle of specific binding to A-T regions of DNA and thus the fluorescence emission in blue at the core. Rhodamine-phalloidin staining is based on the principle of specific binding of phalloidin to actin filaments and thus the emission in red at the cytoplasm. Haematoxylin staining is based on the principle of specific binding of haematoxylin to negatively charged cell components (nucleic acids, membrane proteins, etc.) and thus the emission in purple blue. Eosin staining is based on the principle of specific binding of eosin to cytoplasm and thus the emission in pink (FIG. 30, DAPI/rhodamine-falloidine staining).

Furthermore, cell morphology can be tested by means of histological analysis of some portions of the tissue-engineered construct (e.g., endothelialized scaffold) and inclusion in paraffin and subsequent sectioning by means of microtome.

The functionality of the cells adhered to the scaffold according to steps I-III of the characterisation method subject of the present invention can be verified, for example, by evaluating the gene expression of some typical cell markers. For endothelial cells, typical markers are, for example, Von Willebrand factor (VWF), cluster of differentiation 31 (CD31), vascular cell adhesion molecule 1 (VCAM-1).

The Von Willebrand (VWF) factor is typical of endothelial cells, while the cluster of differentiation 31 (CD31) factor is a typical factor of cell-cell adhesions between endothelial cells. The techniques used to characterise cell functionality can be distinguished into immunofluorescence and immunohistochemistry.

Immunofluorescence assays are based on conjugation between an antibody and the antigens of the VWF protein (found in the cytoplasm) or CD31 protein (found in the cell membrane) and a fluorescence detection system by means of an optical microscope.

Immunohistochemistry assays are based on conjugation between an antibody and the antigens of the VWF protein (found in the cytoplasm) or CD31 protein (found in the cell membrane) and an enzymatic detection system by means of an optical microscope.

Cell functionality is also tested by means of a real-time PCR assay for Von Willebrand factor (VWF) and cluster of differentiation 31 (CD31) factors.

The assay is based on the evaluation of the gene expression and it provides for the extraction of total RNA and—after reverse transcription to cDNA—it is quantified using a specific Taqman Gene Expression Assay (ThermoFisher Scientific) using the real-time PCR technique.

Functional levels for gene expression of the markers listed previously are indicators of good functionality and viability of the cells adhered to the lumen of the scaffold.

As is evident from the results reported in the experimental part, the characterisation step IV of the characterisation method of the present invention shows that steps I-III (and, optionally, phases I.1, II.1-II.2, II.1-II.4, II.1-II.9, II.1-III.3, 2.1-2.11 and/or 3.1-3.13) of said method, in particular the seeding and perfusion method are effective and guarantee a homogeneous, uniform and reproducible seeding, adhesion and growth of viable cells, preferably endothelial cells, along the various lumen sections subject of analysis.

In other words, the characterisation method of the present invention comprising steps I-IV (and, optionally, steps I.1, II.1-II.2, II.1-II.4, II.1-II.9, II.1-III.3, 2.1-2.11 and/or 3.1-3.13) allows to produce, in an easy and reproducible manner, both in laboratory and industrial scale, and to characterise a tissue-engineered construct comprising a scaffold having at least a portion of the lumen lined with at least one functional and preferably continuous cell layer, such as for example a functional and preferably continuous endothelium layer having confluent cells.

Forming an object of the present invention is the tissue-engineered construct comprising a scaffold (21) having at least a portion of the lumen lined with at least one functional cell layer that can be obtained by means of the characterisation method (step I-IV) subject of the present invention. Preferably, said at least one functional cell layer is a layer of functional endothelial cells; more preferably it is a functional and continuous monolayer of endothelial cells having confluent cells. Advantageously, said scaffold can be lined on the outer surface with cells, preferably muscle cells.

Forming an object of the present invention is a method for the in vitro testing of the efficacy, toxicity and/or safety of a medicinal product for human or animal use, preferably for use in the cardiovascular and peripheral vascular region, said method comprising the following steps:
preparing an in-vitro model of a vascular structure comprising the tissue-engineered construct that can be obtained according to the characterisation method according to the present invention (step I-IV), wherein said tissue-engineered construct has functional anatomical and physiological characteristics or, alternatively, it has dysfunctional anatomical and physiological characteristics suitable to simulate a damage or a deformation or a degeneration due to an aneurysm, stenosis, sclerosis plaques, forms of tumours or cardiomyopathies; preferably said vascular structure is selected from among blood vessels, blood ducts and valves of the central or peripheral circulatory system; more preferably said vascular structure is selected from among arteries, veins, capillaries, aortic and mitral valve; followed by
introducing the medicinal product to be tested into said in-vitro model of a vascular structure; preferably the medicinal product is selected from among valves, heart valves, stents, grafts, catheters, bandages, nets or filters; followed by
allowing the circulation—in said in-vitro model—of a vascular structure comprising said medicinal product of a human whole blood sample, artificial blood or derivatives thereof so as to evaluate the behaviour and the interaction of said medicinal product with said human whole blood sample, artificial blood or derivatives thereof.

### Experimental Part

### Evaluation of Cell Viability: Assay for Metabolic Reaction of Cells Adhered to the Scaffold with Resazurin

Methodology 1:

After about 24 hours of adhesion, the seeded scaffold is removed from the grips. Subsequently, the scaffold is sectioned (cutting it) into three areas measuring about 2 cm each depending on the distance from the site of injection of the cell suspension: proximal, medial and distal. Subsequently, each section is divided into 4 parts measuring about 1 cm². 3 samples each representing each region (proximal, medial and distal) of the scaffold with adhered endothelial cells were selected for the assay with resazurin. Each sample is positioned in a well of a 24-well plate and incubated with 1 ml of a 0.02 mg/ml resazurin sodium salt solution with fresh growth medium preferably for 3 hours at 37° C. with 5% of CO₂. The reaction that is developed between the 0.02 mg/ml resazurin sodium salt solution with fresh growth medium and the scaffold sample (with the adhered endothelial cells) is analysed using the arbitrary unit of fluorescence (A.U.) detection at 590 nm by using a spectrofluorometer.

Methodology 2:

Prepare the 0.02 mg/ml resazurin solution in EGM (endothelial growth medium) under a laminar flow hood starting from the 0.2 mg/ml resazurin solution in PBS (phosphate-buffered saline) 1× and EGM medium previously filtered using a 0.1 μm filter. Preheat to 37° C. before use in a thermostated water bath.

Transfer the bioreactor chamber with the scaffold previously seeded with HUVECs under a sterile laminar flow hood inserted thereinto. Disconnect any circuit connected to the bioreactor chamber using clamps and caps for the connectors. Empty the EGM present in the lumen of the scaffold by gravity by tilting the chamber.

Inject a 0.02 mg/ml resazurin solution in EGM into the lumen of the scaffold using the scaffold cell seeding procedure (MMM-05-00).

Prepare the blank for analysis by adding 3 ml of the same resazurin solution into a sterile 15 ml tube that will be placed in the same incubator. Incubate in an incubator at 37° C.+5% CO2 for 30 min. After 30 min, return the bioreactor chamber under a laminar flow hood and empty the lumen of the scaffold by gravity by tilting the chamber and collect the solution in a 15 ml tube.

Move with the samples to the bench for loading the dish for spectrofluorometer reading. Use a p1000 micropipette to transfer 1 ml of each sample into a previously labelled 1.5 ml tube. After spectrofluorometer analysis, store the samples in the special box in the refrigerator at −20° C.

Spectrofluorometer Analysis:

Once the resazurin solution and blank have been collected in a 15 ml tube, use a p200 micropipette to load 100 μl in triplicate of each sample into a 96-well black plate. Read the plate under the spectrofluorometer, setting 510 nm excitation and 590 nm emission fluorescence as detection parameters to read the A.U. (arbitrary unit of fluorescence) values.

### Evaluation of Cell Viability: Quantification of Genomic DNA of Cells Adhered to the Scaffold Using Picogreen

Methodology

The genomic DNA quantification analysis method can be applied to 1 cm² samples of HUVECs adhered to a scaffold section.

Use the Quant-iT PicoGreen dsDNA Reagents and Kits (P7589, Invitrogen) kit and the reagents listed below.

Place the scaffold sections with adhered HUVECs in 1.5 ml tubes and add 180 μl AUT solution and 20 μl of proteinase K, vortex mix using push button mode for 15 sec. Incubate at 56° C. in thermoblock o/n. Add 200 μl of AUL solution and vortex mix with using push button mode for 15 sec. Incubate at 70° C. in a thermoblock for 10 min. Spin in a centrifuge. Add 200 μl of Ethanol (make sure the solvent is not above) 25°) and vortex mix using push button mode for 15 sec.

Centrifuge at 6000×g (8000 rpm) for 1 min. Transfer the lysate to the QIAamp UCP MinElute column inserted into a 2 ml tube.

Centrifuge at 6000×g (8000 rpm) for 1 min. Transfer the column to a new 2 ml tube and add 500 μl of Buffer AUW1.

Centrifuge at 6000×g (8000 rpm) for 1 min. Transfer the column to a new 2 ml tube and add 500 μl of Buffer AUW2.

Centrifuge at 6000×g (8000 rpm) for 1 min. Transfer the column to a new 2 ml tube and centrifuge at 20000×g (12000 rpm) for 3 min. Transfer the column to a previously labelled 1.5 ml tube and add 20 μl of type I H2O directly onto the membrane and incubate at RT for 5 min.

Centrifuge at 20000×g (12000 rpm) for 1 min. Discard the column and store in samples at −20° C.

The following analysis method can be applied to samples of genomic DNA resuspended in H2O.

Preparing the Calibration Curve:

Dilute TE 20× (component B) to TE 1× using type I H2O in a test tube or tube.

Prepare the PicoGreen reagent by diluting 1:200 Quant-iT PicoGreen dsDNA reagent (component A) with TE 1×, prepare at the time of use in a test tube.

Prepare a diluted DNA solution starting from the standard stock solution of dsDNA lambda (100 μg/ml, component C) and prepare the calibration curve using the diluted DNA solution as follows, choosing it in relation to the estimated dsDNA content in the samples.

High-Range Standard Curve:

Prepare a 2 μg/ml dsDNA solution starting from the standard stock solution of dsDNA (100 μg/ml), 1:50 dilution using TE 1× in a 1.5 ml test tube.

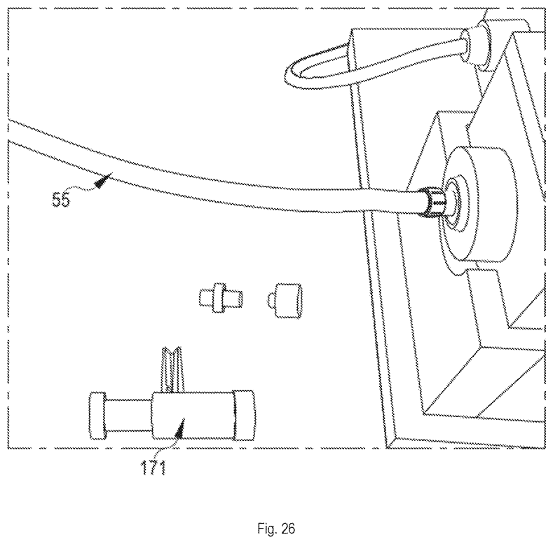

Serially dilute the 2 μg/ml dsDNA solution in 1.5 ml test tubes as shown in FIG. 28.

Low-Range Standard Curve:

Prepare a 50 ng/ml dsDNA solution starting from the 2 μg/ml dsDNA solution, 1:40 dilution using TE 1× in a 1.5 ml test tube.

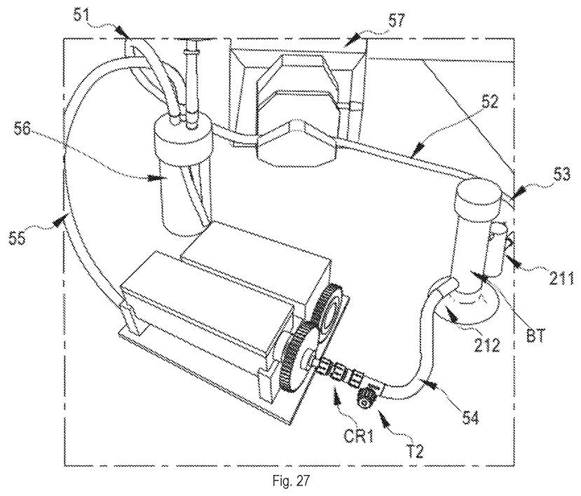

Serially dilute the 50 ng/ml dsDNA solution in 1.5 ml test tubes as shown in FIG. 29.

Sample Analysis:

Load 25 μl in duplicate of each point of the selected curve into the wells of a 96-well black plate. Load 25 μl in duplicate of the TE 1× (blank) solution into wells of the same 96-well black plate. Load 25 μl in triplicate each sample to be quantified in wells of the same 96-well black plate, suitably dilute the samples using TE 1× if necessary.

Add to each loaded well (points of the curve, blank and samples) equal volume, 25 μl, of PicoGreen mixing well.

Incubate the 96-well black plate at RT in the dark for 2-5 min.

Measure fluorescence under the spectrofluorometer setting excitation wavelength at 480 nm and emission wavelength at 520 nm.

Subtract the arbitrary unit of fluorescence (U.A) of the blank sample from that of each of the samples to be quantified and from each sample of the curve.

Use the values obtained to generate a fluorescence standard curve with respect to the DNA concentration, using an Excel file.

Calculate the dsDNA quantity (total ng/ml and ng) of each sample using the fluorescence standard curve.

### Results of the Assays for Metabolic Reaction with Resazurin and Quantification of Genomic DNA of Cells Adhered to the Scaffold using Piccogreen

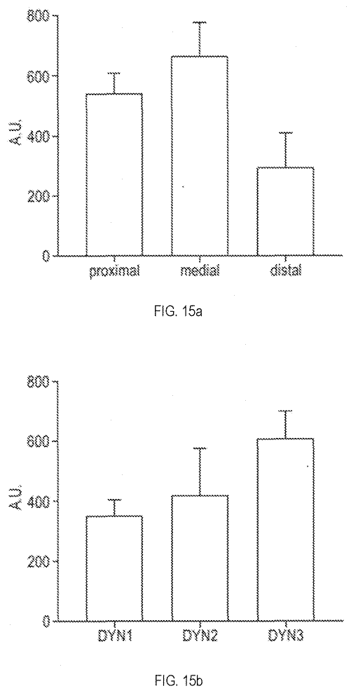

In FIGS. 15A and 15B represented in the chart are the values obtained using the metabolic reaction assay with resazurin according to methodology 1 on samples representing each region (proximal, medial and distal) of a scaffold seeded with endothelial cells and incubated for 24 hours in three different experiments (named DYN1, DYN2 and DYN3). The charts in FIGS. 15A and 15B show a good adhesion and viability of the endothelial cells.

This data was confirmed by the quantification of the genomic DNA (FIGS. 15C and 15D) calculated considering that the genomic DNA content of an endothelial cell is of about 7 pg.

No significant cell viability difference was observed among the various proximal, medial and distal sections of the scaffolds seeded with endothelial cells. In particular, cell viability and the number thereof can be compared along the length (main axis of the scaffold) in the proximal, medial and distal portions thereof.

With the aim of supporting this evidence, costaining was conducted using DAPI (D1306, ThermoFisher scientific) and Rhodamine-Phalloidin (R415, ThermoFisher scientific) on samples representing each region (proximal, medial, distal) of a scaffold seeded with endothelial cells and incubated for about 24 hours. After about 24 hours of culture, these results show that the endothelial cells are viable and distributed on the lumen of the scaffold in a uniform fashion. In particular, these results show an at least 90% cell confluence.

In conclusion, the characterisation step IV of the characterisation method of the present invention shows that steps I-III of said method, in particular the seeding and perfusion method. are effective and guarantee a homogeneous, uniform and reproducible seeding, adhesion and growth of viable endothelial cells long the various lumen sections subject of analysis.

### Evaluation of Cell Morphology gy Staining the Core and the F-Actin Filaments

The following method of analysis is applied to stain the core and the F-actin filaments of HUVECs adhered to scaffold section. It is a destructive method.

This method uses DAPI to stain the core and Rhodamine-phalloidin for cytoplasmic staining.

### Methodology

Place the 1 cm² scaffold sections with adhered HUVECs into wells of a 24-well plate. Use a section of the same type as 1 cm² scaffold seeded with about 2.5*105 cells 24 h before, as an analysis positive control. For each sample proceed as follows.

Carry out 3 consecutive rapid washes using 1× PBS, covering the sample well, using a plastic pasteur pipette. Remove any traces of 1× PBS and—under a fume hood—add 10% neutral buffered formalin, covering each sample well, using a plastic pasteur pipette. Incubate at room temperature for 10 min. Carry out 3 washings using 1× PBS for 5 min, better if under stirring on a stirring plate.

Add the Sudan Black solution in EtOH 70%, covering the sample well, and incubate in the dark at room temperature for 30 min. From now on, handle the samples in the dark.

Move the sample to a well with 1× PBS and carry out repeated washings using 1× PBS until there are no more traces of sudan black. Add the 0.1% Triton solution in 1× PBS, covering the sample well, and incubate in the dark at room temperature for 5 min. Carry out 2 washings using 1× PBS for 5 min, better if under stirring on a stirring plate.

Add the 1% BSA solution in 1× PBS, covering the sample well, and incubate at room temperature for 20 min. Carry out 2 washings using 1× PBS for 5 min, better if under stirring on a stirring plate.

Add the 1:100 Rhodamine-phalloidin solution in 1% BSA in 1× PBS, freshly prepared, using micropipettes covering the sample well, and incubate at room temperature for 20 min. Carry out 3 washings using 1× PBS for 5 min, better if under stirring on a stirring plate.

Add the DAPI 1: 200 solution in 1× PBS, freshly prepared using micropipettes covering the sample well, and incubate at 37° C. for 3 min. Carry out 2 washings using 1× PBS for 5 min, better if under stirring on a stirring plate.

Observe the samples under the fluorescence microscope using a slide and coverslip without a mounting. Observe the nuclear staining through the blue channel and observe the tinting of the actin filaments through the red channel. Store in samples in 1× PBS at 4° C.

### Evaluation of Cell Morphology Using Histological Analysis

The preparation of samples for paraffin embedding and histological evaluation is carried out as described below.

Rinse the tubular section in non-sterile 1× PBS. Immerse the section in 10% formalin in a tube and incubate at ±4° C. for 4 h. Rinse the section in 70% ethanol. Immerse the section in 70% ethanol in a tube and store at ±4° C.

Two sections are obtained from each sample: one transversal measuring 2-3 mm in height and the other longitudinal (remaining part). The preparation continues as follows:
Clarification: since the ethanol, now contained in the preparation after dehydration, cannot be mixed with the substance that will infiltrate it for the embedding step (paraffin), it must be replaced with an intermediate solvent, which can be mixed both with ethanol and with paraffin. The clarifying agent used is xylene.
Embedding: the infiltration steps, to be carried out at a temperature of 56-58 ° C., are as follows:
xylene/paraffin (50%-50%)
pure paraffin (with 2-3 changes)
Sectioning: the microtome is used to obtain slices having a thickness of 5 um.
Dewaxing: the paraffin sections are hydrophobic instead and the paraffin must be removed in order to stain them; therefore, the slides must be treated with:
xylene
xylene/ethanol (50%-50%)
ethanol 100%
ethanol 95%
ethanol 70%
ethanol 50%
distilled water
Staining: haematoxylin eosin
Dehydration: the sections must be dehydrated once again and returned to xylene:
distilled water
ethanol 70%
ethanol 90%
ethanol 95%
ethanol 100% (2 changes)
xylene
Mounting: the coverslip must be firmly fixed to the slide. This is obtained by using natural or synthetic resins which guarantee the perfect adhesion of the two elements to each other and which, upon drying, make the preparation stable and unalterable. (Canada balsam)
Observation: by means of an optical microscope with the following magnifications: 25×; 50×; 100×; 200×; 400× and 1000×

The evaluation of cell morphology and distribution on the inner surface of a scaffold and the cell condition is carried out by staining using haematoxylin and eosin.

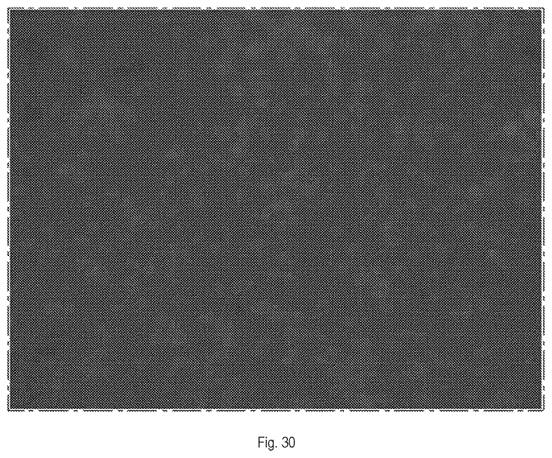

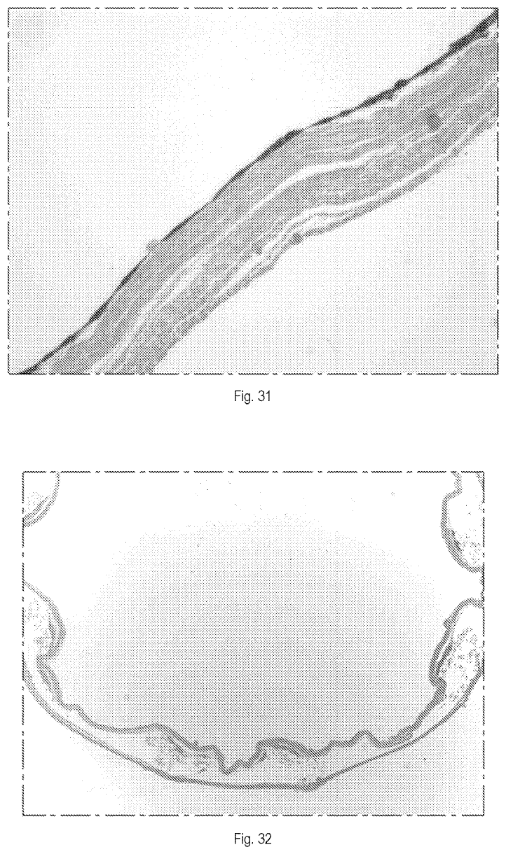

The sample was sectioned and included in paraffin, then 5 μm slices were cut and stained using Haematoxylin and Eosin (FIGS. 31 and 32).

All samples were then examined under a microscope with different magnifications

1. A method for characterising a tissue-engineered construct configured for in vitro testing of medicinal products for human or animal use, said method comprising:
preparing a scaffold having a lumen in a chamber of a bioreactor, to obtain a bioreactor-scaffold system,
seeding at least one portion of the lumen of the scaffold in the bioreactor-scaffold system with a cell culture and allowing adhesion of cells of the cell culture to the scaffold to obtain a seeded bioreactor-scaffold system comprising seeded cells of the cell culture,
stimulating growth and organisation of said seeded cells in the lumen of the scaffold until at least one layer of functional cells adhered to at least one portion of the lumen of the scaffold is formed, to provide the tissue-engineered construct in which the scaffold comprises cells lining at least one portion of the lumen, and
characterising said cells lining at least one portion of the lumen of the scaffold to verify viability, morphology, functionality and/or distribution thereof;
wherein the characterising is performed concurrently or after the stimulating and comprises at least one non-destructive method applied to the cells during or after the stimulating.

2. The method according to claim 1, wherein said cells lining at least one portion of the lumen of the scaffold are endothelial cells selected from cells constituting an endothelium of a vascular tissue.

3. The characterisation method according to claim 1, wherein the lumen of said scaffold is at least partly lined with at least one functional and continuous cell layer.

4. The method according to claim 1, wherein said seeding comprises:
releasing said cell culture in form of a cell suspension comprising a fresh growth medium and cells in a container mounted on a T-shaped connector arranged upstream of the bioreactor by a rotary connector; followed by
releasing said cell culture from the container in the lumen of the scaffold in the bioreactor with a continuous flow such that flow velocity allows said cell suspension to drip into the T-shaped connector without generating air bubbles and to push the air bubbles present in the lumen of the scaffold toward an opening of the T-shaped connector arranged downstream of the bioreactor, allowing outflow thereof.

5. The method according to claim 1, wherein said seeding comprises:
continuously rotating the scaffold along a longitudinal axis thereof for a time interval comprised between 2 and 48 hours with a rotational speed comprised between 0.5 and 5 rpm to allow uniform adhesion of cells to the inner lumen of the scaffold; followed by
incubating the scaffold housed in the bioreactor for a time interval between 2 and 48 hours at a temperature between 20 and 45° C. in presence of CO₂ at 1-10%.

6. The method according to claim 1, wherein the stimulating comprises applying a perfusion method with a hot fresh growth medium having a temperature between 20° C. and 45° C. of the cells present in the lumen of said seeded scaffold; wherein said perfusion method comprises:
connecting an element for removing air bubbles and said seeded bioreactor-scaffold system to a perfusion circuit,
wherein said element for removing air bubbles is inserted upstream of the seeded bioreactor-scaffold system;
filling at least one part of said element for removing air bubbles with said hot fresh growth medium,
wherein said element for removing air bubbles comprises a chamber, a cap for closing said chamber, an access with inflow function and an access with outflow function,
wherein said chamber has a volume and wherein
a first part of said volume is filled with said hot fresh growth medium and
a second part of said volume is filled with air, said second part of said volume having the function of trapping the air bubbles present in said fresh growth medium which flows through said access with inflow function and said access with outflow function; and
allowing the perfusion of the seeded scaffold with said hot fresh growth medium.

7. The method according to claim 1, wherein said at least one non-destructive method is an assay of metabolic reaction of the cells to a reagent.

8. The method according to claim 1, preceding claims, wherein the characterising further comprises at least one destructive method to be applied upon completing the stimulating.

9. The method according to claim 1, wherein said the preparing comprises:
mounting the scaffold in the bioreactor chamber through a scaffold-holder, to obtain the bioreactor-scaffold system, wherein said scaffold is a substantially tubular-shaped polymeric scaffold; and
wherein the seeding comprises:
injecting the fresh growth medium into the lumen of said scaffold fixed on said scaffold-holder arranged inside the bioreactor chamber to obtain a scaffold injected with said growth medium; followed by
adding said fresh growth medium into the bioreactor chamber where said scaffold-holder with the scaffold injected with said growth medium is present; followed by
leaving said growth medium in the lumen of the scaffold and in the bioreactor chamber for a time interval between 1 hour and 18 hours at a temperature between 20° C. and 30° C.; followed by
clearing an internal portion of the lumen of the scaffold and of the bioreactor chamber of the growth medium; followed by
releasing said cell culture in form of a cell suspension comprising a fresh growth medium and cells in a container; followed by
releasing said cell suspension from the container in the lumen of the scaffold; followed by
adding said fresh growth medium into the bioreactor chamber where said scaffold-holder with the scaffold seeded said cell suspension in the lumen is present; followed by
continuously rotating the scaffold along a longitudinal axis thereof for a time interval comprised between 2 and 48 hours with a rotational speed comprised between 0.5 and 5 rpm so as to allow uniform adhesion of cells to the inner lumen of the scaffold, followed by
incubating the scaffold for a time interval between 2 and 48 hours at a temperature between 20 and 45° C. in presence of CO₂ at 1-10%.

10. A tissue-engineered construct comprising a scaffold having at least a portion of the lumen lined with at least one functional cell layer obtained by the method according to claim 1.

11. The tissue-engineered construct according to claim 10, wherein said scaffold is a polymeric scaffold of synthetic origin.

12. The tissue-engineered construct according to claim 10, wherein said scaffold is made of substantially tubular-shaped electrospun silk fibroin.

13. A method for in vitro testing of efficacy and/or toxicity of a medicinal product for human or animal use said method comprising:
preparing an in-vitro model of a vascular structure comprising the tissue-engineered construct according to the method of claim 1, wherein said tissue-engineered construct has functional anatomical and physiological characteristics or, alternatively, it has dysfunctional anatomical and physiological characteristics suitable to simulate a damage or a deformation or a degeneration due to an aneurysm, stenosis, sclerosis plaques, forms of tumours or cardiomyopathies; preferably said vascular structure is selected from among blood vessels, blood ducts and valves of the central or peripheral circulatory system; more preferably said vascular structure is selected from among arteries, veins, capillaries, aortic and mitral valve; followed by
introducing the medicinal product to be tested into said in-vitro model followed by
allowing circulation in said in-vitro model of a human whole blood sample, artificial blood or derivatives thereof so as to evaluate the behaviour and the interaction of said medicinal product with said human whole blood sample, artificial blood or derivatives thereof.

14. The method according to claim 1, wherein the lumen of said scaffold is at least partly lined with at least one monolayer of functional and continuous endothelial cells.

15. The method according to claim 2, wherein the endothelial cells are selected from HAOECs (human aortic endothelial cells), HCAECs (human coronary artery endothelial cells), HMVECs (human dermal microvascular endothelial cells), HUVECs (human umbilical vein endothelial cells).

16. The method according to claim 8, wherein said destructive method is selected from among a DNA quantification assay or a colorimetric assay with 2-(4-amidinophenyl)-1H-indole-6-carboxamidine (DAPI) or rhodamine-phalloidin or haematoxylin or eosin.

17. The tissue-engineered construct of claim 10, wherein said at least one functional cell layer is a layer of functional endothelial cells.

18. The tissue-engineered construct according to claim 10, wherein said scaffold is made of substantially tubular-shaped electrospun silk fibroin.

19. The tissue-engineered construct according to claim 11, wherein said polymeric scaffold of synthetic origin is electrospun silk fibroin or PGA/PLA (polyglycolic acid/polylactic acid) or PGA/PCL (polyglycolic acid/polycaprolactone) copolymers.

20. The method according to claim 13, wherein the method is for in vitro testing of efficacy and/or toxicity of a medicinal product in a cardiovascular and peripheral vascular region, and the medicinal product is selected from among valves, heart valves, stents, grafts, catheters, bandages, nets or filters.
